(19) United States
(12) Patent Application Publication
(10) Pub. No.: US 2018/0171000 A1
(43) Pub. Date: Jun. 21, 2018

(54) RECOMBINANT LIPOPROTEIN MODIFIED BY MONOSIALOTETRAHEXOSYL GANGLIOSIDE AND APPLICATION THEREOF

(71) Applicant: Shanghai Jiaotong University School Of Medicine, Shanghai (CN)

(72) Inventors: XIAOLING GAO, SHANGHAI (CN); MENG HUANG, SHANGHAI (CN); HONGZHUAN CHEN, SHANGHAI (CN); QINGXIANG SONG, SHANGHAI (CN)

(73) Assignee: Shanghai Jiaotong University School Of Medicine

(21) Appl. No.: 15/897,122

(22) Filed: Feb. 14, 2018

Related U.S. Application Data
(63) Continuation of application No. PCT/CN2016/094152, filed on Aug. 9, 2016.

(30) Foreign Application Priority Data
Aug. 14, 2015 (CN) 201510497622.4

(51) Int. Cl. C07K 14/775; A61K 47/42; A61K 47/26; A61K 38/17; A61K 9/00

(52) U.S. Cl. CPC C07K 14/775; A61K 47/42; G01N 33/6896; A61K 38/1716; A61K 9/0043; A61K 47/26

(57) ABSTRACT

The present invention discloses a reconstituted lipoprotein modified by monosialoteterahexosyl ganglioside, an application in preparing a drug carrier thereof, and an application in preparing a drug for treating or preventing a disease associated with Aβ deposition thereof. Specifically, the present invention discloses an application of an appropriate amount of monosialoteterahexosyl ganglioside in modifying the reconstituted lipoprotein to increase an affinity of the reconstituted lipoprotein with amyloid β-protein (Aβ), and facilitating clearance of Aβ. Meanwhile, the reconstituted lipoprotein modified by monosialoteterahexosyl ganglioside is used as a multi-mode nano carrier for preventing and treating central nervous system diseases, and particularly Alzheimer's disease.

### CROSS-REFERENCE TO RELATED APPLICATIONS

This application is a continuation of international Patent Application No, PCT/CN2016/094152 with a filing date of Aug. 9, 2016, designating the United States, now pending, and further claims priority to Chinese Patent Application No. 201510497622.4 with a filing date of Aug. 14, 2015. The content of the aforementioned applications, including any intervening amendments thereto, are incorporated herein by reference.

### TECHNICAL FIELD

The present invention relates to the fields of neuropharmacology and chemical pharmaceuticals, and particularly relates to a reconstituted lipoprotein modified by monosialoteterahexosyl ganglioside, an application in preparing a drug carrier thereof, and an application in preparing a medicine for preventing and treating Alzheimer's disease.

### BACKGROUND OF THE PRESENT INVENTION

Alzheimer's disease (AD), characterized by progressive dementia, is the most common degenerative disorder of the central nervous system that occurs in older people. Its clinical manifestations include continuous deterioration of cognitive and memory functions, progressive decline of the ability of daily living as well as various neuropsychiatric symptoms and behavior disorders. At present, after cardiovascular diseases, cancers and cerebral apoplexy, the incidence of AD is the fourth in the older people, and AD has become the fourth leading cause of death. Along with the acceleration of a population aging process, the incidence of AD is increasing. World Alzheimer's Disease Report points out that, the number of patients with dementia is expected to be nearly doubled every 20 years and may be increased from 36 million in 2010 to 115 million in 2050, and 58% of the patients are living in the lower-middle income countries. Up to 2050, the quantity may be increased to 71%. According to the report, the total expense associated with the dementia reaches 604 billion dollars per year and is about 1% of global gross domestic product (GDP). AD has severely threatened human health and survival quality, and is an increasingly serious public health problem and economic problem.

At present, drugs for clinical treatment of AD are substantially in symptomatic treatment, include acetylcholin esterase (AchE) inhibitors such as tacrine, donepezil, rivastigmine and galanthamine and a gutamic acid NMDA receptor antagonist memantine, and can only improve a decline of learning and memorial functions caused by cholinergic deficiency in a short time, but cannot change the pathological progress of AD. Therefore, there is an urgent need to search and establish a novel prevention and treatment method to achieve an AD disease modification effect.

Senile plaque and neurofibrillary tangles are important pathological features of AD. The main composition of the senile plaque is amyloid β-protein (Aβ), while the neurofibrillary tangles are mainly composed of Tau proteins in hyperphosphorylation. Aβ is a polypeptide composed of 39-43 amino acids, includes two main types, that is, Aβ1-40 and Aβ1-42, and is derived from amyloid precusor protein (APP). Aβ, particularly Aβ-42, has a high aggregation propensity, may rapidly aggregate to form soluble oligomers after secreted by neurons, and further aggregate to form Aβ fibers that is deposited in the brain. A current research clearly points out that, Aβ is a core pathogenic material of AD, wherein Aβ oligomer has the highest neurotoxicity. Excessive production and deposition of Aβ in the brain may cause neuron synapse dysfunction, hyperphosphorylation of the Tau proteins, oxidative stress and secondary inflammatory response, thereby causing aneurodegeneration and neuronal death and finally causing a cognitive disorder. The above description is currently a widely-accepted hypothesis of AD pathogenesis, that is, amyloid β-cascade hypothesis. Therefore, Aβ and aggregates thereof, particularly the oligomer, become the most important disease biomarkers of AD, while how to safely and efficiently decrease Aβ level in the brain becomes a key challenge for preventing and treating AD.

Deposition of Aβ is caused by the broken equilibrium between the production and the elimination of Aβ. Therefore, the decrease of the production or the promotion of the clearance is key means for decreasing Aβ level in the brain. Since 1990s, a first discussion is to decrease the production of Aβ by inhibiting activities of key enzymes (β-secretase and γ-secretase) for the production of Aβ. However, since the β-secretase and γ secretase simultaneously participate in the metabolic processes of numerous substrates, severe adverse reactions may be produced by simply inhibiting the activities of the β-secretase and the γ-secretase due to an interference of normal physiological functions of the neurons, γ secretase inhibitors (including semagacestat in Eli Lilly and Company and avagacestat in Bristol-Myers Squibb) failed in succession in clinical trials, so that the research and development enthusiasm of an Aβ precursor protein (APP) metabolic regulator drops to a freezing point.

The incidence of late onset patients accounts for 90%-95% of AD patients. The speed of Aβ production in brains of these patients is the same as that of normal people, while the rate of Aβ clearance is obviously lower in AD patients than that in normal control. Therefore, to accelerate the clearance of Aβ in the brain becomes the most important direction for preventing and treating AD. At present, passive immunotherapy is the most frequently studied with respect to the clearance of Aβ. An antibody with specificity and high affinity to Aβ is adopted in the passive immunotherapy, and Aβ can form complex with antibodies via their Fc fragment and then been uptake and cleared by macrophage. However, some important problems exist in the passive immunotherapy as follows: (1) the Aβ-antibody immune complex induces the following adverse reactions that: Aβ, as an autoantigen, forms the immune complex with an antibody entering the brain, and may induce secondary immune response in terms of central nervous system inflammations and damage of vascular walls, thereby causing intra-cerebral inflammations, cerebral capillary bleeding, vasogenic cerebral edema and other adverse reactions; and (2) current effective antibodies are all specific antibodies targeting the amino terminal of Aβ, and since a sequence of Aβ amino terminal is located at an extracellular fragment of the APP, the antibodies resistant to Aβ amino terminal may bind to the APP of the neurons and cause an immune attack to normal neurons. Meanwhile, failures of clinical tests of Aβ antibodies including bapineuzumab and its resemblance indicate that, although Aβ can be effectively removed by Aβ antibodies, the cognitive improvement of AD patients is poor. Possible reasons may be that the secondary pathological processes caused by Aβ aggregation, such as synapse dysfunction, neuron loss and the like, once initiated by Aβ, may independently exacerbate the pathological process of AD; however clinically, when the AD patient has disease symptoms, the nervous system functions have already suffered from irreversible damage; and at this moment, the pathological features of the AD patient are difficult to be improved if the focus is only on the clearance of Aβ. Therefore, there is an urgent need to explore an AD multi-mode therapeutic strategy for not only facilitating the clearance of Aβ but also protecting the neurons or inhibiting the development of other secondary pathological processes.

Nanocarrier has been applied for developing a multi-mode therapeutic strategy for the treatment of tumors, acquired immunodeficiency syndrome and other diseases. With respect to the multi-mode therapeutic method of AD, the nano carrier needs high brain-blood barrier (BBB) penetration, or by virtue of a drug delivery manner which avoids the brain-blood barrier, as well as high affinity to Aβ, to realize targeted drug delivery.

### SUMMARY OF PRESENT INVENTION

The first purpose of the present invention is to provide a reconstituted lipoprotein modified by monosialoteterahexosyl ganglioside (GM1) for increasing affinity between the reconstituted lipoprotein and Aβ, increasing concentrations of the reconstituted lipoprotein and a drug in the central nervous system and protecting neurons while facilitating clearance of Aβ.

The second purpose of the present invention is to provide an application of the reconstituted lipoprotein modified by monosialoteterahexosyl ganglioside in preparing a drug carrier.

The third purpose of the present invention is to provide an application of the reconstituted lipoprotein modified by monosialoteterahexosyl ganglioside in preparing a drug for treating or preventing a disease associated with deposition of Aβ.

In order to realize the first purpose, the present invention discloses a technical solution as follows: the reconstituted lipoprotein modified by monosialoteterahexosyl ganglioside is characterized by comprising monosialoteterahexosyl ganglioside, lipid and an apolipoprotein, wherein the monosialoteterahexosyl ganglioside accounts for 1%-30% of a total lipid molar fraction.

As a preferred solution, the monosialoteterahexosyl ganglioside accounts for 1%-20% of the total lipid molar fraction, preferably 1%-18%, 1-15%, 1-10%, 2%-25%, 2%-20%, 2%-18%, 2%-15%, 3%-25%, 3%-20%, 3%-18%, 3%-15%, 4%-25%, 4%-20% and 5%-20%.

As a preferred solution, the mass of the apolipoprotein accounts for 1-60% of prescription content.

As a preferred solution, the mass of the apolipoprotein accounts for 1-50% of the prescription content.

As a preferred solution, the mass of the apolipoprotein accounts for 1-40%, 1-30%, 1-25% and 1-20% of the prescription content, preferably 2-60%, 3-60%, 4-60% and 5-60%, preferably 2-50%, 3-50%, 4-50%, 5-50%, and more preferably 2-40%, 3-40%, 4-30%, 5-30%, 2-25%, 3-25%, 4-25%, 5-30%, 5-25%, 5-20%, 14%.

As a preferred solution, the lipid is one or more of egg lecithin, fabaceous lecithin, phosphatidylcholine, phosphatidyl ethanolamine, phosphatidylserine, phosphatidyl glycerol, phosphatidylinositol, phosphatidic acid, cardiolipin, lysophosphatide, sphingosine, ceramide, sphingomyelin, cerebroside, cholesterol, cholesteryl ester, glyceride and derivatives thereof.

As a preferred solution, the lipid does not include cholesterol or cholesteryl ester.

As a preferred solution, the apolipoprotein is one or more of ApoE and mimic peptides thereof, ApoA-I and mimic peptides thereof, ApoA-II and mimic peptides thereof and ApoC and mimic peptides thereof, particularly preferably ApoE and mimic peptides thereof. The ApoE includes ApoE2, ApoE3 and ApoE4.

A particle size range of the reconstituted lipoprotein modified by monosialoteterahexosyl ganglioside is 1-500 nm, and preferably 5-100 nm.

Preparation methods of the reconstituted lipoprotein include a thin-film rehydration method, an injection method, a multiple emulsion method, a melting method, a freeze-drying method, a reverse evaporating method, a high pressure homogenization method or an ultrasonic method and a Ca2+ fusion method.

In order to realize the second purpose of the present invention, the present invention discloses a technical solution as follows: the reconstituted lipoprotein modified by monosialoteterahexosyl ganglioside is applied to preparing the drug carrier. The drug may be a drug for treating any disease, and includes one or more of small-molecule chemicals, macromolecular polypeptides, proteins and gene drugs. The gene drugs include nucleic acid drugs such as nucleic acids, nucleotides, nucleosides and bases and derivatives and analogs thereof, etc., and include siRNA, microRNA or antisense nucleic acids, etc.

As a preferred solution, the drug carrier is a carrier for nasal delivery. Excipients of a nasal delivery preparation include one or more of water, sodium chloride, potassium chloride, sodium carbonate, sodium phosphate, sodium tetraborate, sodium acetate, sodium bicarbonate, methylcellulose, ethyecellulose, hydroxypropyl methylcellulose, polyvinylpyrrolidone, polyvinyl alcohol, hydroxyethyl cellulose, hydroxy propyl cellulose, hydroxypropyl ethyl cellulose, sodium carboxymethylcellulose, polyethylene glycol, polymethyl methacrylate, polycarbophil, gelatin, alginic acid, polyethylene acid, polyoxyethylene, sodium chondroitin sulfate, sodium hyaluronate, chitosan, sodium hydrogen sulfite, sodium hydrogen sulfate, sodium thiosulfate, benzalkonium chloride, chlorbutanol, thiomersalate, phenylmercuric acetate, phenylmercuric nitrate, methyl parahydroxybenzoates, phenethyl alcohol, mannitol, glucose, glycerin and xylitol.

As a preferred solution, the drug is a drug for treating or preventing a disease of the central nervous system, such as a drug for treating or preventing the Alzheimer's disease.

In order to realize the third purpose of the present invention, the present invention discloses a technical solution as follows: the reconstituted lipoprotein modified by monosialoteterahexosyl ganglioside is applied to preparing a drug for treating or preventing the disease associated with the deposition of Aβ.

As a preferred solution, the disease associated with the deposition of Aβ is the Alzheimer's disease.

As a preferred solution, the drug is given via the nasal route.

According to the reconstituted lipoprotein modified by monosialoteterahexosyl ganglioside in the present invention, the lipid is any other lipid except the GM1, and the total lipid refers to all lipids including the GM1.

The disease associated with the deposition of Aβ in the present invention refers to a deposition of Aβ and a cognitive disorder caused by diseases of Alzheimer's disease, Parkinson's disease, Huntington's disease. Creutzfeldt-Jakob disease and diabetes, cerebral apoplexy and the like.

The present invention has the following advantages that:

1) the present invention first proposes the reconstituted lipoprotein modified by monosialoteterahexosyl ganglioside, and gives the content of components with excellent effects;

2) compared with an unmodified reconstituted lipoprotein, the reconstituted lipoprotein modified by the GM1 has obviously increased affinity with Aβ and increased treatment effect for AD;

3) the reconstituted lipoprotein modified by the GM1 also has the drug-carrying characteristic, and the drug can be carried to the reconstituted lipoprotein modified by the monosialoteterahexosyl ganglioside, thereby achieving an effect of treating multiple diseases, particularly the disease associated with the deposition of Aβ or the disease of the central nervous system;

4) particularly, compared with the unmodified reconstituted lipoprotein, the reconstituted lipoprotein modified by the GM1 has higher nasal mucosa absorption efficiency and intra-cerebral delivery characteristics during nasal delivery;

5) the GM1 enables the reconstituted lipoprotein to be stable and difficult to aggregate; the cholesterol may be not added into the lipids, but an equivalent and even better effect can be achieved than the lipids added with the cholesterol, and such an improvement may overcome the defects of drug leakage from the reconstituted lipoprotein caused by in vivo esterification of the cholesterol, and avoid the possible adverse reactions produced by excessive intra-cerebral delivery of the cholesterol, and the like; and

6) more importantly, an appropriate amount of GM1 may obviously decrease the dose of the apolipoprotein ApoE in the total formulation but in the meanwhile maintain or enhance the drug loading capacity, the binding affinity to Aβ, therapeutic effects and the like.

### DESCRIPTION OF THE DRAWINGS

FIG. 1 illustrates the transmission electron microscope image of (A) a reconstituted lipoprotein unmodified by the monosialoteterahexosyl ganglioside; (B) a reconstituted lipoprotein modified by the monosialoteterahexosyl ganglioside, scale bar: 20 nm;

FIG. 2 illustrates (A) the binding curves of reconstituted lipoprotein with Aβ1-42 monomer; (B) the binding curves of a reconstituted lipoprotein with Aβ1-42 oligomer; (C) the binding curves of reconstituted lipoprotein modified by monosialoteterahexosyl ganglioside with Aβ1-42 monomer; and (D) the binding curves of the reconstituted lipoprotein modified by monosialotetetrahexosyl ganglioside with A 1-42 oligomer;

FIG. 3 illustrates (A) Aβ1-42 uptake, (B) Aβ1-42 degradation in primary microglial cells in the presence of the reconstituted lipoprotein or the reconstituted lipoprotein modified by monosialoteterahexosyl ganglioside, and (C) co-location of the reconstituted lipoprotein or the reconstituted lipoprotein modified by monosialoteterahexosyl ganglioside with Aβ1-42 in primary microglial cells; *p<0.05,**p<0.01,***p<0.0001 represent there are significant differences between the reconstituted lipoprotein modified by monosialoteterahexosyl ganglioside and the reconstituted lipoprotein;

FIG. 4 illustrates the cellular uptake of the reconstituted lipoprotein modified by monosialoteterahexosyl ganglioside in a 16HBE cell line;

FIG. 5 shows effects of a reconstituted lipoprotein and a reconstituted lipoprotein modified by monosialoteterahexosyl ganglioside on the brain clearance of Aβs following nasal drug delivery in a Aβ intra-cerebral injected mouse model: (A) percentage of the amount of free Aβ1-42 in the mouse brain (that is, the amount of degraded Aβ1-42) to the total injection amount; and (B) the amount of Aβ1-42 in the mouse brain transported to periphery via blood brain barriers, represented as the brain excretion index; n=3-5, *p<0.05,**p<0.01, significantly different from that of the control group;

FIG. 6 illustrates degradation of Aβ in brains following intravenous injection of the reconstituted lipoprotein or the reconstituted lipoprotein modified by monosialoteterahexosyl ganglioside in a Aβ intra-cerebral injected mouse model, represented by percentage of the amount of free Aβ1-42 in the mouse brain (that is, the amount of degraded Aβ1-42) to the total injection amount;

FIG. 7 illustrates the neuroprotective effect the NAP-loaded reconstituted lipoprotein modified by monosialoteterahexosyl ganglioside; primary neurons are co-incubated with Aβ1-42 oligomers and the NAP fusion peptide solution, the NAP-loaded reconstituted lipoprotein modified by monosialoteterahexosyl ganglioside, and the reconstituted lipoprotein modified by monosialoteterahexosyl ganglioside or the culture medium (the blank control) for 48 h; (A) the count of neuron cells, (B) mean neurite length and (C) mean branch-point count are recorded. *p<0.05,**p<0.01,***p<0001, ****p<0.0001 significantly different with that of the Aβ1-42 only control;

FIG. 8 illustrates influences of two-week daily injection of the reconstituted lipoprotein, the reconstituted lipoprotein modified by monosialoteterahexosyl ganglioside and the NAP-loaded reconstituted lipoprotein modified by monosialoteterahexosyl ganglioside on the escape latency of AD model mice investigated by the Morris water maze test; *p<0.05,***p<0.001 indicate that there is a significant difference from the blank control group; and #p<0.05 indicates that there is a significant difference from the reconstituted lipoprotein group modified by monosialoteterahexosyl ganglioside.

### DETAILED DESCRIPTION OF PREFERRED EMBODIMENTS

The present invention is further described below in combination with specific embodiments. If not specially described, experimental methods used in embodiments below are all conventional methods. If not specially described, materials, reagents and the like used in embodiments below may be all commercially available. It should be understood that, these embodiments are used for describing the present invention only, rather than limiting a scope of the present invention.

### Embodiment 1. Method for Increasing Monodispersity of a Reconstituted Lipoprotein Through the Modification with Monosialoteterahexosyl Ganglioside

(1) Preparation

steps: doping monosialoteterahexosyl ganglioside which accounts for 5%, 10% and 20% of a molar ratio of total lipids into dimyristoyl phosphatidylcholine which accounts for 95%, 90% and 80% of the molar ratio of the total lipids, dissolving with chloroform, performing decompression evaporation to remove the organic solvent, hydrating lipid membranes in phosphate buffer at a pH of 7.4, and ultrasonically homogenizing to obtain liposome (the total mass of the lipid of 4 mg) modified by the monosialoteteterahexosyl ganglioside; and adding 0.8 mg of ApoE, slightly mixing to be uniform, and incubating in a orbital shaker at 100 rpm at 37° C. for 36 h, thereby obtaining the reconstituted lipoprotein modified by the monosialoteterahexosyl ganglioside.

(2) Characterization

Particle sizes and zeta potentials are determined by dynamic laser scattering. Results show that, the particle size of liposome modified by the monosialoteterahexosyl ganglioside which is not incubated with the ApoE is 55.17±5.11 nm, the particle size of ApoE-incubated reconstituted lipoproteins modified by the monosialoteterahexosyl ganglioside (the monosialoteterahexosyl ganglioside accounting for 5%, 10% and 20% of the molar ratio of the total lipids) is decreased to be less than 25 nm, and the zeta potentials are −14.20±0.66 mV, −20.20±0.36 mV and −26.41±0.42 mV, respectively, while the particle size of the reconstituted lipoprotein unmodified by the monosialoteterahexosyl ganglioside is 24.64±3.59 nm, and the zeta potential is −8.06±0.78 mV.

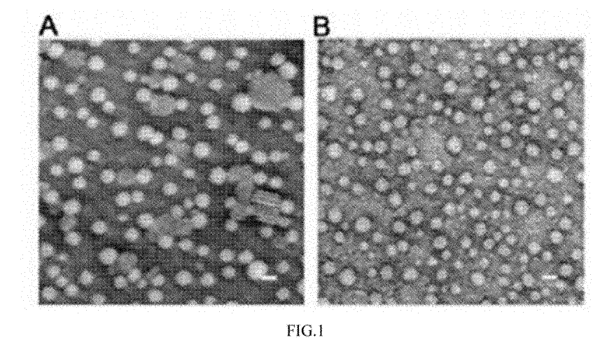

Phosphotungstic acid negative staining is performed, and morphology is observed by a transmission electron microscope. Results show that (FIG. 1), the reconstituted lipoprotein unmodified by the monosialoteterahexosyl ganglioside has the particle size of about 20 nm and is partially stacked into a silkworm pupa shape, while the reconstituted lipoprotein modified by the monosialoteterahexosyl ganglioside also has the particle size of about 20 nm but has better dispersity, and stacked lipoproteins are not observed. The reasons may be that the zeta potential on the surface of the reconstituted lipoprotein modified by the monosialoteterahexosyl ganglioside is more negative, particles are difficulty aggregated due to electrostatic repulsion and the monodispersity is better.

### Embodiment 2. Method for Increasing Binding Affinity to Aβ with a Reconstituted Lipoprotein Through the Modification with Monosialoteterahexosyl Ganglioside

Preparation

steps: doping monosialoteterahexosyl ganglioside which accounts for 5%, 10% and 20% of a molar ratio of total lipids into dipalmitoyl phosphatidyl choline which accounts for 95%, 90% and 80% of the molar ratio of the total lipids (the total mass of the lipid is 4 mg), adding 0.8 mg of ApoE, and preparing a reconstituted lipoprotein modified by the monosialoteterahexosyl ganglioside by using the same method in embodiment 1;

doping cardiolipin which accounts for 5% of the molar ratio of the total lipids into the dipalmitoyl phosphatidyl choline which accounts for 95% of the molar ratio of the total lipids (the total mass of the lipid is 4 mg), adding 0.8 mg of ApoE, and preparing a reconstituted lipoprotein modified by the cardiolipin according to the above method;

and doping sulfatide which accounts for 10% of the molar ratio of the total lipids into the dipalmitoyl phosphatidyl choline which accounts for 90% of the molar ratio of the total lipids (the total mass of the lipid is 4 mg), adding 0.8 mg of ApoE, and preparing a reconstituted lipoprotein modified by the sulfatide according to the above method.

(2) A surface plasmon resonance (SPR) experiment verifies A-binding affinity to the reconstituted lipoprotein modified by the monosialoteterahexosyl ganglioside.

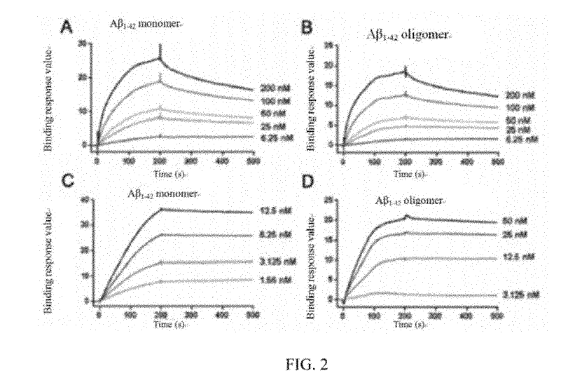

An Aβ monomer or oligomer is fixed on a CM5 chip via amine coupling: activating the surface of the chip by using 0.2 M EDC and 0.05 M NHS, diluting Aβ monomer or oligomer in sodium acetate buffer solution at pH 4.0, enabling the concentration of Aβ to be 23 μM, injecting at a speed of 30 μl/min, and blocking with ethanol amine at pH 8.5; and directly blocking with the ethanol amine after the reference channel is activated. The affinity test is detected in a dual-channel mode: diluting the reconstituted lipoprotein in 10 mM PBS at a pH of 7.4, and injecting into the reference channel and the Aβ-fixed channel at a speed of 30 μl/min, wherein contact time is 100 s or 300 s, and dissociation time is 400 s. Results are analyzed using the Biacore T200Evaluation Software program, and affinity value is calculated using 1:1 binding model. The results show that, the reconstituted lipoprotein modified by the monosialoteterahexosyl ganglioside accounting for 5%, 10% and 20% of the molar ratio of the total lipids is of high-binding affinity (FIG. 2) with Aβ1-42 monomer or oligomer, and affinity constants KD of the reconstituted lipoprotein modified by the monosialoteterahexosyl ganglioside accounting for 5% of the molar ratio of the total lipids with Aβ1-42 monomer or oligomer are calculated by thedynamic method, are (1.7±1.90)×10-10 and (1.51±0.02)×10-10 M, respectively, and are respectively increased by 58 times and 62 times compared with affinity between the unmodified reconstituted lipoprotein and the Aβ1-42 monomer and oligomer (the affinity constants KD of (9.97±2.81)×10-9 and (9.47±4.37)×10-9, respectively), which indicates that the affinity characteristic between the reconstituted lipoprotein and Aβ is increased by the modification of the monosialoteterahexosyl ganglioside.

Affinity of lipidosome modified by the monosialoteterahexosyl ganglioside which is not incubated with ApoE, and ApoE alone with the Aβ1-42 monomer and oligomer is also detected. Results show that, the affinity constants of the liposome modified by the monosialoteterahexosyl ganglioside which is not incubated with the ApoE, with the Aβ1-42 monomer and oligomer are respectively 1.50×10-8 M and 4.04×10-8 M, the affinity constant of the ApoE alone with the Aβ1-42 monomer is 2.95×10-8 M, and the affinity constant of the ApoE protein with the Aβ1-42 oligomer is difficult to be calculated due to the weak binding signal. The affinity constants of the reconstituted lipoprotein modified by the 5% of cardiolipin prepared according to the same method with Aβ1-42 monomer and oligomer are (14.96±2.51)×10-9 and (6.06±4.66)×10-9 M, respectively; and the affinity constants of the reconstituted lipoprotein modified by the 10% of sulfatide prepared according to the same method with Aβ1-42 monomer and oligomer are 68.26×10-9 and (18.15±7.34)×10-9 M, respectively. The above results indicate that the affinity of the reconstituted lipoprotein to Aβ is specifically increased by the modification of the monosialoteterahexosyl ganglioside.

TABLE 1
Comparison of affinity of different reconstituted lipoproteins to Aβ

| | Aβ₁₋₄₂ monomer | | | Aβ₁₋₄₂ oligomer | | |
|---|---|---|---|---|---|---|
| | Kₐₛₛ | | | Kₐₛₛ | | |
| | (M⁻¹s⁻¹) | K_{diss} (s⁻¹) | K_{D}(M) | (M⁻¹s⁻¹) | K_{diss} (s⁻¹) | K_{D}(M) |
| Reconstituted | (11.38 ± 3.27) × | (1.72 ± 1.69) × | (0.17 ± 0.190) × | (6.58 ± 4.98) × | (9.93 ± 7.30) × | (1.51 ± 0.02) × |
| lipoprotein modified by | 10⁵ | 10⁻⁴ | 10⁻⁹ | 10⁵ | 10⁻⁵ | 10⁻¹⁰ |
| 5% of | | | | | | |
| monosialoteterahexosyl | | | | | | |
| ganglioside | | | | | | |
| Reconstituted | (7.48 ± 0.32) × | (11.15 ± 1.40) × | (14.96 ± 2.51) × | (19.57 ± 14.7) × | (8.44 ± 0.21) × | (6.06 ± 4.66) × |
| lipoprotein modified by | 10⁴ | 10⁻⁴ | 10⁻⁹ | 10⁴ | 10⁻⁴ | 10⁻⁹ |
| 5% of cardiolipin | | | | | | |
| Reconstituted | 4.19 × 10³ | 2.86 × 10⁻³ | 68.26 × | (5.30 ± 1.79) × | (8.89 ± 2.72) × | (18.15 ± 7.34) × |
| lipoprotein modified by | | | 10⁻⁹ | 10⁴ | 10⁻⁴ | 10⁻⁹ |
| 10% of sulfatide | | | | | | |
| Unmodified | (12.55 ± 6.08) × | (11.66 ± 2.53) × | (9.97 ± 2.81) × | (9.30 ± 3.66) × | (8.00 ± 0.66) × | (9.47 ± 4.37) × |
| reconstituted | 10⁴ | 10⁻⁴ | 10⁻⁹ | 10⁴ | 10⁻⁴ | 10⁻⁹ |
| lipoprotein | | | | | | |

### Embodiment 3. Method for Increasing Microglial Cells-Mediated Uptake and Degradation Following the Treatment with Reconstituted Lipoprotein Modified by Monosialoteterahexosyl Ganglioside

(1) Preparation

steps: adding monosialoteterahexosyl ganglioside which accounts for 5% of a molar ratio of total lipids into a mixture of phosphatidylcholine and phosphatidic acid accounting for 95% of the molar ratio of the total lipids (the total mass of the lipid is 4 mg), adding 0.4 mg of ApoE, and preparing a reconstituted lipoprotein modified by the monosialoteterahexosyl ganglioside by using the same method in embodiment 1.

(2) Facilitation of a Uptake in Primary Microglial Cells by the Reconstituted Lipoprotein Modified with Monosialoteterahexosyl Ganglioside

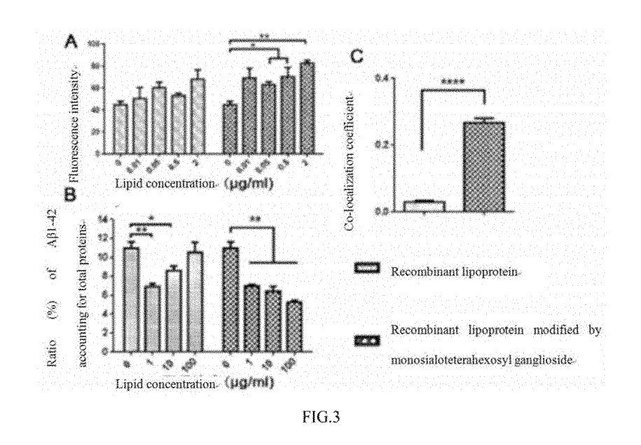

steps: adding FAM fluorescently-labeled Aβ1-42 into 96-well plates cultured with microglial cells, diluting the reconstituted lipoprotein and the reconstituted lipoprotein modified with the monosialoteterahexosyl ganglioside with DMEM and adding into 96-well plates cultured with primary microglial cells until the final concentrations are 0, 0.01, 0.05, 0.5 and 2 μg/mL, wherein the final concentration of the FAM fluorescently-labeled Aβ1-42 is 2 μg/mL; co-incubating in a 37° C. cell incubator for 4 h; fixing in 3.7% of formaldehyde at 37° C. for 10 min, performing Hoechst nucleus staining for 8 min, washing with PBS for 3 times, and performing shooting and quantitative analysis by virtue of a high-content TargetActivation program. Results are shown in FIG. 3A, and the uptake of the primary microglial cells to the FAM fluorescently-labeled Aβ1-42 is obviously increased by the reconstituted lipoprotein modified by monosialoteterahexosyl ganglioside.

(3) Investigating the influence of reconstituted lipoprotein modified by monosialoteterahexosyl ganglioside to Aβ degradation in primary microglial cells via ELISA.

steps: diluting Aβ1-42 to 4 μg/ml with DMEM, adding into the 24-well plates cultured with microglial cells, adding the reconstituted lipoprotein or the reconstituted lipoprotein modified by monosialoteterahexosyl ganglioside diluted by the DMEM to the final concentrations 0, 1, 10, 100 μg/mL, wherein the final concentration of tAβ1-42 is 2 μg/mL; and incubating at 37° C. for 4 h, lysing and scraping cells, and performing subsequent detection or preserving at −80° C.;

taking a 1 mg/ml of human Aβ1-42 standard stock solution stored at −80° C., diluting to 0, 6.25, 12.5, 25, 50, 100 and 200 pg/ml with dilution buffer to serve as a standard curve; diluting a sample by 80 times with the dilution buffer; and performing the experiment and result determination according to an ELISA product specification, and applying BCA analysis to determine the total protein amount in cell lysate. The result is represented as the ratio of the Aβ1-42 concentration in the cell lysate to the total protein concentration. The result is shown in FIG. 3B, and indicates that, the reconstituted lipoprotein modified by monosialoteterahexosyl ganglioside can increase Aβ1-42 degradation in the primary microglial cells in a concentration-dependent manner, and due to an addition of the GM1, the content of the ApoE may be obviously decreased.

### Embodiment 4. The Uptake of Reconstituted Lipoprotein Modified by Monosialoteterahexosyl Ganglioside by an In Vitro Nasal Mucosa Cell Model

A 16HBE cell line serves as the in vitro nasal mucosa cell model, and cellular uptake of the reconstituted lipoprotein modified by the monosialoteterahexosyl ganglioside are investigated.

(1) Preparation

The reconstituted lipoprotein modified by the monosialoteterahexosyl ganglioside is prepared according to the same method in embodiment 1.

(2) Cellular Uptake of the Reconstituted Lipoprotein Modified by Monosialoteterahexosyl Ganglioside in the 16HBE Cell Line.

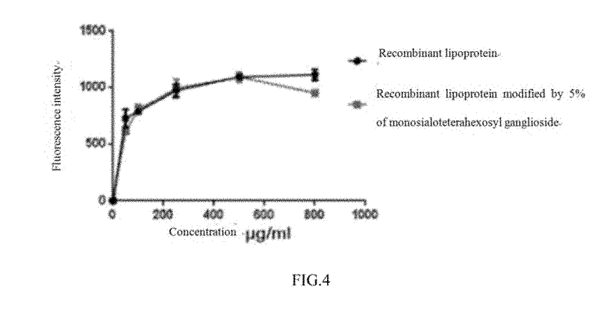

Steps: inoculating 16HBE into 96-well plates, culturing for 24 h, and conducting the experiment; diluting the formulation to 50, 100, 250, 500 and 800 μg/m with DMEM, adding the material into the above 96-well plates, and incubating at 37° C. for 4 h; fixing the cells in 3.7% of formaldehyde, performing Hoechst nucleus staining after PBS washing, and performing quantitative analysis using a high-content analysis. The result indicates that, the cellular uptake amount of the reconstituted lipoprotein modified by monosialoteterahexosyl ganglioside accounting for 5% of the molar ratio of the total lipids is equivalent to that of an unmodified reconstituted lipoprotein (FIG. 4).

### Embodiment 5. Intra-Cerebral Delivery Efficiency of a Reconstituted Lipoprotein Modified by Monosialoteterahexosyl Ganglioside Via Nasal Delivery

Reconstituted lipoproteins modified at a monosialoteterahexosyl ganglioside modification degree increased from 5% of the molar ratio of the total lipids to 10%, 20%, 30% and 40% are prepared according to the same method in embodiment 1.

125I is labeled onto the reconstituted lipoprotein modified by monosialoteterahexosyl ganglioside via Bolton-Hunter method. Results show that, following nasal administration in ICR mice, the maximal concentration (Cmax) of the 125I-labeled reconstituted lipoprotein modified by monosialoteterahexosyl ganglioside accounting for 5% of the molar ratio of the total lipids in the cortex and hippocampus is 0.0434% ID/g, and is 75% higher than that of the 125I-labeled unmodified reconstituted lipoprotein; and the AUC all in the cortex and hippocampus is 0.2956% ID/g·h, which is 85% higher than that of the unmodified reconstituted lipoprotein; and the AUC all in blood is 9.8650% ID/g·h, which is 80% higher than that of the unmodified reconstituted lipoprotein. However, the AUCCortex+Hippocampus/AUCblood of the two preparations is close to each other, which indicates that the reconstituted lipoprotein modified by monosialoteterahexosyl ganglioside has higher nasal mucosa absorption efficiency than the unmodified reconstituted lipoprotein. But along with an increase of the monosialoteterahexosyl ganglioside modification degree from 5% to 10%, 20%, 30% and 40%, the intra-cerebral delivery characteristic of the formulations is not obviously increased, but shows a certain decline trend. Intra-cerebral distribution of a high-density 125I-labeled reconstituted lipoprotein modified by monosialoteterahexosyl ganglioside accounting for 20% of the molar ratio of the total lipids is slightly higher than that of an unmodified high-density reconstituted lipoprotein (increased by 25.9%), the intra-cerebral distribution of a 125I-labeled high-density reconstituted lipoprotein modified by monosialoteterahexosyl ganglioside accounting for 30% of the molar ratio of the total lipids is equivalent to that of the unmodified high-density reconstituted lipoprotein (102.3%), and the intra-cerebral distribution of a high-density 125I-labeled reconstituted lipoprotein modified by monosialoteterahexosyl ganglioside accounting for 40% of the molar ratio of the total lipids is lower than that of the unmodified high-density reconstituted lipoprotein (87.2%).

TABLE 2
Pharmacokinetic parameters of the unmodified reconstituted
lipoprotein and the reconstituted lipoprotein modified by
monosialoteterahexosyl ganglioside in mouse following nasal delivery

| | | | Reconstituted lipoprotein | |
|---|---|---|---|---|
| | Unmodified | | modified by | |
| | reconstituted | | monosialoteterahexosyl | |
| | lipoprotein | | ganglioside | |
| | Cortex + hippocampus | | Cortex + hippocampus | |
| | Blood | | Blood | |
| Tₘₐₓ (h) | 4 | 4 | 2 | 4 |
| Cₘₐₓ (% ID/g) | 0.0248 | 0.8074 | 0.0434 | 1.333 |
| AUCₐₗₗ (% ID/g) | 0.1596 | 5.4945 | 0.2956 | 9.8650 |

| | | |
|---|---|---|
| AUC_{Cortex+Hippocampus}/ | 0.0291 | 0.0300 |
| AUC_{blood} | | |

### Embodiment 6. Method for Facilitating Clearance of Aβ in Mouse Brains by a Reconstituted Lipoprotein Modified with Monosialoteterahexosyl Ganglioside

(1) Preparation

steps: adding monosialoteterahexosyl ganglioside which accounts for 5% of a molar ratio of total lipids into a mixture of phosphatidylcholine and phosphatidylcholine which accounts for 95% of the molar ratio of the total lipids (a total mass of the lipid is 4 mg), adding 0.8 mg of ApoE and preparing a reconstituted lipoprotein modified by the monosialoteterahexosyl ganglioside by using the same method in embodiment 1.

(2) Evaluation of the Influence of the Reconstituted Lipoprotein Modified by the Monosialoteterahexosyl Ganglioside on Aβ Clearance in Brain Via a Mouse Intra-Cerebral Injection Model

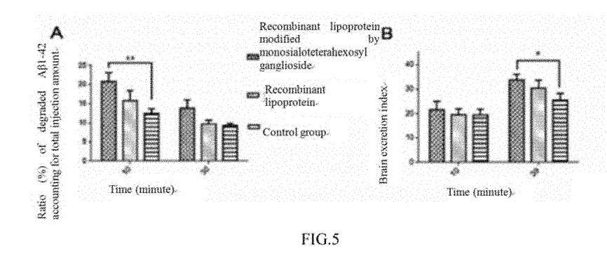

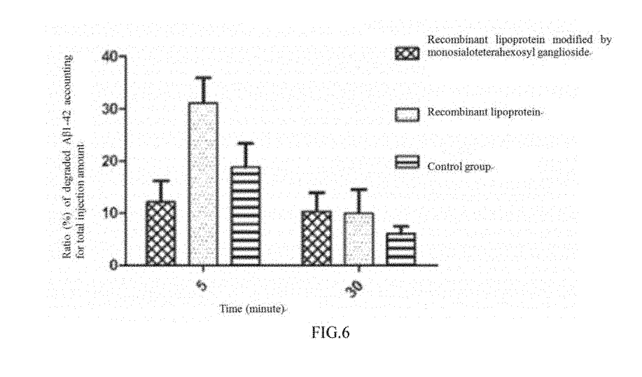

steps: randomly dividing ICR mice into 3 groups, administrating the mice with reconstituted lipoproteins or reconstituted lipoproteins modified by monosialoteterahexosyl ganglioside through nasal delivery or intravenous injection, and giving the control animals with PBS; performing intracerebral A□ injection at 30 min after drug delivery, i.e., mixing 125I-labeled Aβ1-42 and 14C-containing inulin ([14C]inulin), diluting with artificial cerebrospinal fluid, and accurately injecting the material into the mouse hippocampal area; sacrificing the mice at 10 min and 30 min after the intracerebral injection, and collecting the mice brains; weighing half of the brain tissues, detecting the radiation quantity of the 125I-labeled Aβ1-42 on a γ-ray counter, adding 10% of trichloroacetic acid (TCA) at the volume of 3 times of the weight of brain tissues to precipitate the tissues, centrifuging to remove the supernatant, and detecting the radiation intensity of the un-degraded 125I-labeled Aβ1-42. FIG. 5A shows that the reconstituted lipoprotein modified by monosialoteterahexosyl ganglioside obviously facilitates degradation of Aβ1-42 in the brain at a short time after nasal delivery; and FIG. 5B shows that the reconstituted lipoprotein modified by monosialoteterahexosyl ganglioside obviously facilitates the brain-to-peripheral clearance of Aβ1-42 following nasal delivery. By contrast, the reconstituted lipoprotein modified by monosialoteterahexosyl ganglioside does not show an obvious facilitation effect on the degradation of Aβ1-42 in the brain immediately (5 min) after intravenous injection, while the unmodified high-density reconstituted lipoprotein slightly facilitates the degradation of Aβ1-42 in the brain at this time point (FIG. 6). It is supposed that, shortly following intravenous injection, the intra-cerebral distribution of the reconstituted lipoprotein modified by monosialoteterahexosyl ganglioside could be less than that of the unmodified high-density reconstituted lipoprotein, and the Aβ degradation facilitation effect of the reconstituted lipoprotein modified by monosialoteterahexosyl ganglioside needs be achieved at the longer time points (e.g., 30 min). In contrast, the reconstituted lipoprotein modified by monosialoteterahexosyl ganglioside increase the degradation of Aβ1-42 and facilitate brain-to-periphery transport of Aβ1-42 shortly after nasal administration.

### Embodiment 7. Neuroprotective Effect of a Drug-Loaded Reconstituted Lipoprotein Modified by Monosialoteterahexosyl Ganglioside

(1) Preparation

steps: adding monosialoteterahexosyl ganglioside which accounts for 5%, 10% and 20% of a molar ratio of total lipids into dimyristoyl phosphatidylcholine which accounts for 95%, 90% and 80% of the molar ratio of the total lipids, dissolving with chloroform, performing decompression evaporation to remove the organic solvent, hydrating lipid membranes in phosphate buffer at pH 7.4, and ultrasonically homogenizing to obtain liposome (a total mass of the lipid of 4 mg) modified by the monosialoteterahexosyl ganglioside; adding 0.05-1 mg of NAP fusion peptide, incubating overnight at 4° C., adding 0.5-5 mg of ApoE3, and continuously performing ultrasonic treatment for 50 min; and cooling to a room temperature, and incubating overnight, thereby obtaining the NAP-loaded reconstituted lipoprotein modified by the monosialoteterahexosyl ganglioside.

(2) Neuroprotective Effect of the NAP-Loaded Reconstituted Lipoprotein Modified by Monosialoteterahexosyl Ganglioside

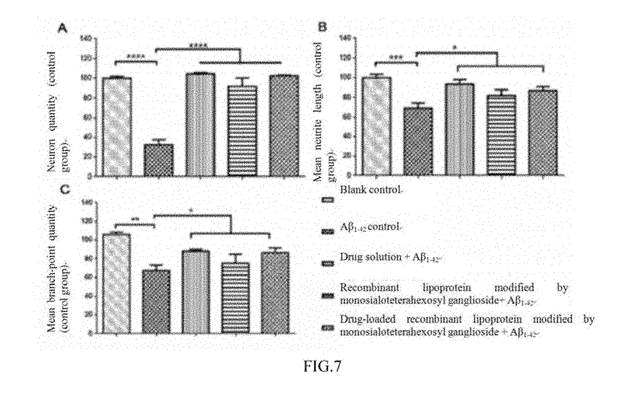

steps: diluting Aβ1-42 oligomer to 20 μM with neuron culture medium, adding the oligomer into 96-well plates cultured with primary neurons, adding the NAP fusion peptide solution, the NAP-loaded reconstituted lipoprotein modified by monosialoteterahexosyl ganglioside or a reconstituted lipoprotein modified by monosialoteterahexosyl ganglioside, enabling the final concentration of the Aβ1-42 oligomer to be 10 μM, and only adding the neuron culture medium into the blank control group; co-incubating at 37° C. for 48 h, fixing the cells with PBS containing 37% of formaldehyde, adding a TritonX-100 permeable membrane for 15 min, blocking at 37° C. by using PBS with 1% of BSA for 30 min, adding the MAP2 antibody to incubate overnight at 4° C., adding an Alexa Fluor 488 fluorescent-conjugated secondary antibody, incubating at 37° C. for 1 h, and performing Hoechst nucleus staining; and detecting the primary neuron cells by using a high-content analysis, and analyzing the data via the NeuroProfiling program. Results are shown in FIGS. 7A-7C, and the NAP-loaded reconstituted lipoprotein modified by monosialoteterahexosyl ganglioside obviously reverses Aβ1-42 oligomer-induced neurotoxicity, thereby increasing the quantity of neurons, the mean neurite length and the mean branch-point counts.

### Embodiment 8. Method for Decreasing Expression of β-Secretase in SH-SY5Y Cells by BACE-siRNA-Loaded Reconstituted Lipoprotein Modified by Monosialoteterahexosyl Ganglioside

steps: preparing a reconstituted lipoprotein modified by monosialoteterahexosyl ganglioside according to the same method in embodiment 1, enabling BACE-siRNA to link with cholesterol, and inserting into a lipid membrane; culturing the SH-SY5Y cells to reach confluence of 80%, and transfecting the cells with 1 μM siRNA; and detecting the expression of β-secretase in the cells by Western blot at 48 h after transfection, and statistically quantifying the gray value of the band by virtue of Image J software. Results indicate that the BACE-siRNA-loaded reconstituted lipoprotein modified by monosialoteterahexosyl ganglioside decreases the expression of the f-secretase in the SH-SY5Y cells by 40%.

### Embodiment 9. Method for Alleviating Inflammations of Microglial Cells Caused by a Cu2+-Aβ Complex by Curcumin-Loaded Reconstituted Lipoprotein Modified by Monosialoteterahexosyl Ganglioside

steps: dissolving curcumin and lipids in a chloroform-methanol mixed solvent according to a certain ratio, and preparing a reconstituted lipoprotein modified by monosialoteterahexosyl ganglioside according to the same method in embodiment 1; incubating Cu2+-Aβ at 37° C. at a molar ratio of 1:1 for 24 h to prepare a complex; adding primary microglial cells into 5 μM of the Cu2+-Aβ complex to incubate for 24 h, increasing content of TNF-α by 100%, and adding the Cu2+-Aβ complex and the curcumin-loaded reconstituted lipoprotein modified by monosialoteterahexosyl ganglioside to simultaneously incubate the primary microglial cells, wherein the content of the TNF-α is equivalent to that in an untreated group. It is indicated that, the curcumin-loaded reconstituted lipoprotein modified by monosialoteterahexosyl ganglioside effectively alleviates the inflammations of the microglial cells caused by the Cu2+-Aβ complex.

### Embodiment 10. Method for Synergistically Improving Spatial Learning and Memory Ability of AD Model Animals by the Reconstituted Lipoprotein Modified by Monosialoteterahexosyl Ganglioside and the Loaded Drug

(1) Preparation

An NAP-loaded reconstituted lipoprotein modified by monosialoteterahexosyl ganglioside is prepared according to the same method in embodiment 7.

(2) Synergistic Improvement of the Spatial Learning and Memory Ability of the AD Model Animals by the Reconstituted Lipoprotein Modified by Monosialoteterahexosyl Ganglioside and the Loaded Drug

Improvement effects of the reconstituted lipoprotein modified by monosialoteterahexosyl ganglioside and the NAP-loaded reconstituted lipoprotein modified by monosialoteterahexosyl ganglioside on the space learning-memory capabilities of the AD model animals are investigated by the Morris water maze test. The AD model mice are randomly divided into groups; each group includes 7-8 mice; and the mice are subjected to nasal delivery according to the following manners: the blank control group and the AD model group: giving PBS solution, 10 μl/mouse per day; the AD model group: giving PBS solution, 10 μl/mouse per day; the drug solution group: giving an NAP fusion peptide solution, 24 μg/kg per day; the reconstituted lipoprotein group: giving there constituted lipoprotein solution, 5 mg/kg per day (lipid concentration); the reconstituted lipoprotein modified by monosialoteterahexosyl ganglioside group: giving the lipoprotein modified by monosialoteterahexosyl ganglioside solution, 5 mg/kg per day (lipid concentration); and the drug-loaded reconstituted lipoprotein modified by monosialoteterahexosyl ganglioside group: giving the solution of the NAP-loaded reconstituted lipoprotein modified by monosialoeterahexosyl ganglioside, 5 mg/kg per day (lipid concentration). The mice are subjected to continuous nasal delivery per day for 14 days.

The Morris water maze has a pool diameter of 120 cm, a height of 50 cm, a water depth of 25 cm and a water temperature of 22±1° C., Four start points are equally divided along a circumference of the pool; a circular pool is equally divided into four quadrant areas I, II, III and IV by virtue of connecting lines of the four start points, and a 9 cm black platform is arranged in a center of the quadrant area I. The platform is about 1 cm lower than a water surface. A bottom of the pool, the platform and four walls are painted black by food dyes, so that the platform is invisible. Swimming trajectories of the mice are monitored and recorded by the Morris water maze video analysis system 2.0. A hidden platform test is used for training and measuring the spatial learning abilities of the mice, lasting for 5 days. The platform is fixed in the center of the quadrant area I; the mice are put into water towards pool walls from the start points in the quadrant areas I, II, III and IV according to a random principle; and routes of the mice from a moment of entering the water and searching to a moment of finding and climbing up the black platform, the needed time (escape latency), swimming speeds and the like are monitored and recorded by a computer. If a mouse does not find the platform within 60s, the mouse should be led to the platform and stopped for 30s, and the escape latency is recorded as 60s. Each mouse is trained by 4 times per day, and a training interval is 30s each time.

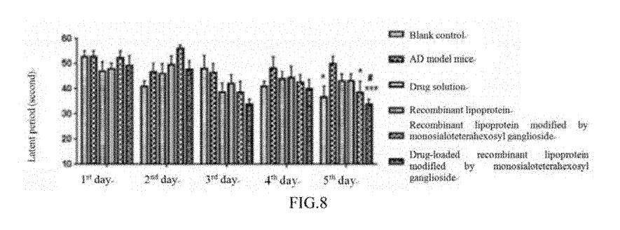

The results are shown in FIG. 8. In a training process of 5 days, the escape latencys of the drug solution group and the reconstituted lipoprotein are not obviously decreased. The escape latency of the reconstituted lipoprotein modified by monosialoteterahexosyl ganglioside in the training on the 5th day is obviously lower than that of the AD model group, which indicates that the reconstituted lipoprotein modified by monosialoteterahexosyl ganglioside has a certain improvement effect on the learning-memory abilities of the AD model mice. After 14-day nasal delivery of the NAP-loaded reconstituted lipoprotein modified by monosialoteterahexosyl ganglioside, the escape latency of the AD model mice in a spatial probe test have a decline trend on the 3rd day. In the training on the 5th day, compared with the AD model group, the escape latency is obviously shortened (33.9±2.1 s), and compared with a non-drug-loaded reconstituted lipoprotein modified by monosialoteterahexosyl ganglioside, the escape latency is shortened by 12%. The results indicate that, the drug-loaded reconstituted lipoprotein modified by monosialoteterahexosyl ganglioside could further enhance a disease modifying effect on the AD by virtue of the multi-mode effect of the reconstituted lipoprotein modified by monosialoeterahexosyl ganglioside and the loaded neuroprotective peptide.

### Embodiment 11. Method for Maintaining High Aβ Binding Affinity of Reconstituted Lipoproteins Via the Modification with Monosialoteterahexosyl Ganglioside Even at Decreased Amount of Apolipoproteins

steps adding monosialoteterahexosyl ganglioside which accounts for 10% of a molar ratio of total lipids into dimyristoyl phosphatidylcholine (4 mg) which accounts for 90% of the molar ratio of the total lipids, adding ApoE3 accounting for 2.5%, 5%, 10% or 20% of lipid mass (0.1, 0.2, 0.4 and 0.8 mg), and preparing the reconstituted lipoprotein modified by monosialoteterahexosyl ganglioside. A surface plasmon resonance (SPR) experiment is performed to detect the Aβ-binding affinity of the reconstituted lipoprotein modified by the monosialoteterahexosyl ganglioside. The A□ binding affinity constants of the reconstituted lipoproteins modified by the monosialoteterahexosyl ganglioside with the ApoE3 content accounting for 2.5%, 5%, 10% and 20% of the lipid mass to Aβ are 0.19×10-9M, 0.64×10-9M, 0.80×10-9M and 0.52×10-9M, respectively. The evidences indicate that, when monosialoteterahexosyl ganglioside is added, even if the mass ratio of apolipoprotein is greatly decreased, the binding affinity of the reconstituted lipoproteins to Aβ may be still maintained or enhanced.

### Embodiment 12. Method for Increasing Brain Delivery of Aβ Polypeptide Vaccine by the Reconstituted Lipoprotein Mediated by Modification of Monosialoteterahexosyl Ganglioside

steps: preparing the reconstituted lipoprotein modified by monosialoteterahexosyl ganglioside according to the above method, adding an 125I-labeled Aβ polypeptide vaccine and co-incubating for 5 min; performing intravenous injection of the mixture, collecting the brain tissues at 1 h after injection, and detecting the radioactive intensity of the 125I-labeled Aβ polypeptide vaccine in the brain by a γ-ray counter. Results show that, compared with single injection of Aβ polypeptide vaccine, the co-incubation with reconstituted lipoprotein modified by monosialoteterahexosyl ganglioside increases the intra-cerebral amount of Aβ polypeptide vaccine by 76.47%, while the co-incubation with unmodified reconstituted lipoprotein only increases the intra-cerebral amount of Aβ polypeptide vaccine by 11.44%. The result indicates that, by virtue of simple co-incubation, the reconstituted lipoprotein modified by monosialoteterahexosyl ganglioside can effectively increase the intra-cerebral delivery of a polypeptide drug with an intra-cerebral transport characteristic, and such effect could be more striking for the brain delivery of those drugs that are difficult to enter the brain.

The above only describes preferred embodiments of the present invention. It should be noted that, those ordinary skilled in the art may make several improvements and modifications on premise of not deviating from a principle of the present invention. These improvements and modifications should also be regarded as a protection scope of the present invention.

We claim:

1. A reconstituted lipoprotein modified by monosialoteterahexosyl ganglioside, comprising monosialoteterahexosyl ganglioside, lipid and an apolipoprotein, wherein the monosialoteterahexosyl ganglioside accounts for 1%-30% of a total lipid molar fraction.

2. The reconstituted lipoprotein modified by monosialoteterahexosyl ganglioside according to claim 1, wherein the monosialoteterahexosyl ganglioside accounts for 1%-20% of the total lipid molar fraction.

3. The reconstituted lipoprotein modified by monosialoteterahexosyl ganglioside according to claim 1, wherein the mass of the apolipoprotein accounts for 1-60% of prescription content.

4. The reconstituted lipoprotein modified by monosialoteterahexosyl ganglioside according to claim 3, wherein the mass of the apolipoprotein accounts for 1-50% of the prescription content.

5. The reconstituted lipoprotein modified by monosialoteterahexosyl ganglioside according to claim 1, wherein the lipid is one or more of egg lecithin, fabaceous lecithin, phosphatidylcholine, phosphatidyl ethanolamine, phosphatidylserine, phosphatidyl glycerol, phosphatidylinositol, phosphatidic acid, cardiolipin, lysophosphatide, sphingosine, ceramide, sphingomyelin, cerebroside, cholesterol, cholesteryl ester, glyceride and derivatives thereof.

6. The reconstituted lipoprotein modified by monosialoteterahexosyl ganglioside according to claim 1, wherein the lipid does not include cholesterol or cholesteryl ester.

7. The reconstituted lipoprotein modified by monosialoteterahexosyl ganglioside according to claim 1, wherein the apolipoprotein is one or more of ApoE and mimic peptides thereof, ApoA-I and mimic peptides thereof, ApoA-II and mimic peptides thereof and ApoC and mimic peptides thereof.

8. An application of the reconstituted lipoprotein modified by monosialoteterahexosyl ganglioside of claim 1 in preparing a drug carrier.

9. The application of the reconstituted lipoprotein modified by monosialoteterahexosyl ganglioside in preparing a drug carrier according to claim 8, wherein the drug comprises one or more of small-molecule chemicals, macromolecular polypeptides, proteins and gene drugs.

10. The application of the reconstituted lipoprotein modified by monosialoteterahexosyl ganglioside in preparing a drug carrier according to claim 8, wherein the drug is a drug for treating or preventing a disease of the central nervous system.

11. An application of the reconstituted lipoprotein modified by monosialoteterahexosyl ganglioside of claim 1 in preparing a drug for treating or preventing a disease associated with deposition of Aβ.

12. The application of the reconstituted lipoprotein modified by monosialoteterahexosyl ganglioside in preparing the drug for treating or preventing a disease associated with deposition of Aβ according to claim 11, wherein the disease associated with the deposition of Aβ is the Alzheimer's disease.

13. The application of the reconstituted lipoprotein modified by monosialoteterahexosyl ganglioside in preparing the drug for treating or preventing a disease associated with deposition of Aβ according to claim 11, wherein the drug is nasal administrated.
